(19) United States
(12) Patent Application Publication
(10) Pub. No.: US 2006/0024260 A1
(43) Pub. Date: Feb. 2, 2006

(54) Cosmetic composition comprising a water-dispersible linear sulphonic polyester and a modified guar gum, methods employing this composition and uses

(76) Inventors: Cecile Bebot, Clichy (FR); Ludivine Laurent, Clichy (FR); Sabine Vrignaud, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/177,334

(22) Filed: Jul. 11, 2005

Related U.S. Application Data
(60) Provisional application No. 60/620,335, filed on Oct. 21, 2004.

(30) Foreign Application Priority Data
Jul. 29, 2004 (FR) 04 08396

(51) Int. Cl. A61K 8/85

(52) U.S. Cl. 424/70.11

(57) ABSTRACT

The present disclosure relates to a cosmetic composition comprising, in a cosmetically acceptable medium, at least one water-dispersible linear sulphonic polyester and at least one modified guar gum, methods employing this composition, and uses thereof.

This application claims benefit of U.S. Provisional Application No.60/620,335, filed Oct. 21, 2004, the contents of which are incorporated herein by reference. This application also claims benefit of priority under 35 U.S.C. § 119 to French Patent Application No. 04 08396, filed Jul. 29, 2004, the contents of which are also incorporated by reference.

The present disclosure relates to a cosmetic composition comprising, in a cosmetically acceptable medium, at least one water-dispersible linear sulphonic polyester and at least one modified guar gum, and the methods employing this composition. The present disclosure also relates to the use, for fixing the hairstyle, of a cosmetic composition comprising, in a cosmetically acceptable medium, at least one water-dispersible linear sulphonic polyester and at least one modified guar gum.

Cosmetic compositions for the shaping and/or form retention of the hairstyle can be in the form of a spray and comprise a solution, generally alcoholic, and one or more components, referred to as fixing components, which are generally polymeric resins, the role of which may be to form connections between the hairs or to sheath the hairs. These fixing components are often formulated as a mixture with various cosmetic adjuvants.

Cosmetic compositions containing fixing components, as described, are generally packaged either as a pump-action spray or in an appropriate aerosol container pressurized using a propellant. The aerosol system comprises, on the one hand, a liquid phase (or fluid) and, on the other hand, a propellant.

It is also possible to use such styling compositions in the form of gels, creams or foams.

Once applied to the hair, it is intended that the fixing components in such styling compositions will make it possible to fix the hair. However, the polymers commonly used as fixing agents in styling compositions often do not make it possible to retain the shaping of the hairstyle when the latter is brought into contact with water for a long period of time, such as, for example, when brought into contact with rain or perspiration or during bathing: sea bathing or bathing in a swimming pool, and the like.

The present inventors have discovered, surprisingly, that the use of a cosmetic composition comprising, in a cosmetic acceptable medium, at least one water-dispersible linear sulphonic polyester and at least one modified guar gum makes it possible to fix and/or shape hairstyles and/or to retain the shaping of hairstyles when the latter are brought into contact with water for a prolonged period of time. Within the meaning of the present disclosure, this phenomenon is referred to as “resistance to water”.

The term “prolonged period of time”, as used herein, is understood to mean being brought into contact with water for a minimum period of time of one minute, such as 10 minutes or, for example, 20 minutes.

The compositions disclosed herein also make it possible to obtain a hairstyle which is resistant to atmospheric moisture.

The compositions according to the present disclosure make possible good fixing and/or good form retention of the hair, that is to say, a styling effect which may last throughout the day, indeed even for several days, and/or which may exhibit good resistance to water, such as good resistance to repeated bathing. These compositions may also exhibit the advantage of being removed on shampooing. Overall, these compositions make it possible to confer good cosmetic properties.

Disclosed herein, therefore, is a cosmetic composition comprising, in a cosmetically acceptable medium, at least one water-dispersible linear sulphonic polyester and at least one modified guar gum.

Also disclosed is a method for the shaping or form retention of the hairstyle in which the above-described cosmetic composition is employed.

Further disclosed herein is the use of a cosmetic composition comprising, in a cosmetically acceptable medium, at least one water-dispersible linear sulphonic polyester and at least one modified guar gum as a styling composition for the fixing and/or the form retention of the hair, such as when it is brought into contact with water for a prolonged period of time, for instance in the case of rain or perspiration or when bathing: such as sea bathing or bathing in a swimming pool. According to this embodiment, the use of the composition makes it possible to obtain a water-resistant hairstyle (wherein “hairstyle” refers to the shaping of the hair). Further, in this embodiment, the cosmetic composition can be provided in a form chosen from lotions, sprays, foams, gels and creams.

Other characteristics, aspects and advantages of the present disclosure will become even more clearly apparent on reading the description and examples which follow.

The term “styling cosmetic composition”, within the meaning of the present disclosure, is understood to mean a composition for the shaping or form retention of a hairstyle.

The cosmetically acceptable medium used in the compositions disclosed herein is an aqueous, aqueous/alcoholic or alcoholic medium optionally comprising at least one additional organic solvent. The alcohol may be a monohydroxylated alkanol chosen from lower C₁-C₄ alcohols, such as ethanol, isopropanol, tert-butanol or n-butanol; in at least one embodiment, the alcohol used is ethanol.

The concentration of alcohol in the compositions according to the present disclosure ranges from 0 to 50%, such as from 0 to 10% or from 0 to 5%, by weight with respect to the total weight of the composition.

The additional organic solvents which can be used in the compositions according to the present disclosure include polyols, such as propylene glycol, polyol ethers and their mixtures.

The concentration of additional organic solvent in the compositions according to the present disclosure ranges from 0 to 30%, such as from 0 to 20%, by weight with respect to the total weight of the composition.

Water-Dispersible Linear Sulphonic Polyesters

As stated above, the composition according to the present disclosure comprises a water-dispersible linear sulphonic polyester. The term “water-dispersible linear sulphonic polyester” is understood herein to mean any sulphonic polyester exhibiting an ability to form a dispersion, that is to say a two-phase system, where the first phase is formed of finely divided particles uniformly distributed in the second continuous phase.

The term “sulphonic polyester” is understood herein to mean copolyesters obtained by polycondensation of at least one dicarboxylic acid or of one of its esters, of at least one diol and of at least one difunctional sulphoaryldicarboxyl compound substituted on the aromatic ring by an —SO₃M group in which M is chosen from a hydrogen atom and a metal ion, such as Na⁺, Li⁺ or K⁺.

The water-dispersible linear sulphonic polyesters used in the context of the present disclosure exhibit a weight-average molecular mass ranging from 1000 to 60,000, such as from 4000 to 20,000, as determined by gel permeation chromatography (or GPC).

The glass transition temperature (Tg) of these sulphonic polyesters may range from 10° C. to 100° C. In at least one embodiment, the Tg of the at least one water-dispersible linear sulphonic polyester used is greater than or equal to 50° C. The glass transition temperature (Tg) is measured by differential scanning calorimetry (DSC) according to Standard ASTM D3418-97.

The water-dispersible linear sulphonic polyesters useful herein are described in more detail, for example, in U.S. Pat. Nos. 3,734,874, 3,779,993, 4,119,680, 4,300,580, 4,973,656, 5,660,816, 5,662,893 and 5,674,479.

In at least one embodiment, the water-dispersible linear sulphonic polyesters comprise at least units derived from isophthalic acid, from a salt of sulphoaryldicarboxylic acid and from diethylene glycol. For example, the sulphonic polyesters may be obtained from isophthalic acid, sodium salt of sulphoisophthalic acid, diethylene glycol and 1,4-cyclohexanedimethanol.

Mention may be made, as examples of water-dispersible linear sulphonic polyesters, of those known under the INCI name Diglycol/CHDM/Isophthalates/SIP and sold under the trade names “Eastman AQ Polymer” (AQ35S, AQ38S, AQ55S, AQ48 Ultra) by Eastman Chemical.

The concentration of water-dispersible linear sulphonic polyester(s) used in the compositions according to the present disclosure ranges from 0.1 to 40%, such as from 1 to 30%, or from 5 to 25%, by weight with respect to the total weight of the composition.

Modified Guar Gums

The composition according to the invention comprises at least one modified guar gum.

The term “modified guar gum”, within the meaning of the present disclosure, is understood to mean -guar gums alkylated by at least one C₁₋₈ alkyl group, guar gums hydroxyalkylated by at least one C₁₋₈ hydroxyalkyl group or guar gums acylated by at least one C₁₋₈ acyl group.

For example, the modified guar gum may be hydroxypropylated guar gum, such as that sold under the name Jaguar HP 105 by Rhodia.

The concentration of modified guar gum(s) used in the compositions according to the present disclosure ranges from 0.05 to 10%, such as from 0.1 to 5% or from 0.5 to 3%, by weight with respect to the total weight of the composition.

Advantageously, the water-dispersible linear sulphonic polyester/modified guar gum ratio ranges from 0.01 to 100, such as from 1 to 75 or from 2 to 50.

he compositions according to the present disclosure can also comprise at least one additional cosmetic adjuvant, such as those mentioned below.

Fixing Polymers

With respect to fixing polymers as the at least one additional cosmetic adjuvant, any anionic, cationic, amphoteric or nonionic fixing polymer, and mixtures thereof, used in the art can be used in the compositions according to the present disclosure.

The additional fixing polymers may be soluble in the cosmetically acceptable medium or may be insoluble in this same medium and, in this case, used in the form of dispersions of solid or liquid polymer particles (e.g., latex or pseudolatex).

Anionic Fixing Polymers

The anionic fixing polymers that may be useful are polymers comprising groups derived from carboxylic, sulphonic or phosphoric acid and have a number-average molecular mass ranging from 500 to 5,000,000.

The carboxyl groups may be contributed by unsaturated mono- or dicarboxylic acid monomers such as those corresponding to the formula:

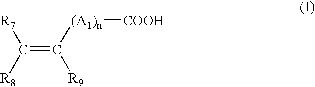

in which

n is an integer from 0 to 10,

A₁ is a methylene group, optionally connected to the carbon atom of the unsaturated group, or to the neighboring methylene group when n is greater than 1, via a heteroatom, such as oxygen or sulphur,

R₇ is chosen from a hydrogen atom, a phenyl group, and a benzyl group,

R₈ is chosen from a hydrogen atom, a lower alkyl group, and a carboxyl group, and

R₉ is chosen from a hydrogen atom, a lower alkyl group, a —CH₂—COOH group, a phenyl group, and a benzyl group.

In the abovementioned formula, a “lower alkyl group,” means, in at least one embodiment, a group having 1 to 4 carbon atoms, such as methyl and ethyl groups.

In at least one embodiment, the anionic fixing polymers are chosen from homopolymers or copolymers of acrylic and methacrylic acid or their salts, copolymers of crotonic acid, copolymers of C₄-C₈ monounsaturated carboxylic acids or anhydrides, polyacrylamides comprising carboxylate groups, homopolymers or copolymers comprising sulpho groups, anionic polyurethanes and anionic grafted silicone polymers.

Suitable anionic fixing polymers comprising carboxyl groups according to the present disclosure may be chosen from, but are not limited to:

A) Homo- or copolymers of acrylic or methacrylic acid or their salts, such as the products sold under the names Versicol® E or K by Allied Colloid and Ultrahold® by BASF, the copolymers of acrylic acid and of acrylamide sold in the form of their sodium salts under the names Reten 421, 423 or 425 by Hercules or the sodium salts of polyhydroxycarboxylic acids.

B) Copolymers of acrylic or methacrylic acid with a monoethylenic monomer, such as ethylene, styrene, vinyl esters or esters of acrylic or methacrylic acid, optionally grafted onto a polyalkylene glycol, such as polyethylene glycol, and optionally crosslinked. Such polymers are disclosed, for example, in French Patent No.1 222 944 and German Application No. 2 330 956, the copolymers of this type comprising, in their chain, an optionally N-alkylated and/or -hydroxyalkylated acrylamide unit, such as disclosed in Luxembourgian Patent Applications Nos. 75370 and 75371 or provided under the name, Quadramer by American Cyanamid. Mention may also be made of copolymers of acrylic acid and of C₁-C₄ alkyl methacrylate and terpolymers of vinylpyrrolidone, of acrylic acid and of C₁-C₂₀ alkyl methacrylate, for example lauryl methacrylate, such as that sold by ISP under the name Acrylidone® LM, and methacrylic acid/ethyl acrylate/tert-butyl acrylate terpolymers, such as the product sold under the name Luvimer® 100 P by BASF. Mention may also be made of the methacrylic acid/acrylic acid/ethyl acrylate/methyl methacrylate copolymers in aqueous dispersion sold under the name Amerhold® DR 25 by Amerchol.

C) Copolymers of crotonic acid, such as those comprising, in their chain, vinyl acetate or propionate units and optionally other monomers, such as allyl or methallyl esters, vinyl ethers or vinyl esters of a linear or branched saturated carboxylic acid comprising a long hydrocarbon chain, such as those comprising at least 5 carbon atoms, it optionally being possible for these polymers to be grafted or crosslinked, or alternatively another monomer chosen from vinyl, allyl and methallyl esters of α- or β-cyclic carboxylic acids. Such polymers are disclosed, inter alia, in French Patents Nos. 1 222 944,1 580 545, 2 265 782, 2 265 781, 1 564 110 and 2 439 798. Commercial products coming within this class are the Resins 28-29-30, 26-13-14 and 28-13-10 sold by National Starch.

D) Copolymers of C₄-C₈ monounsaturated carboxylic acids or anhydrides chosen from:

copolymers comprising
(i) at least one acid or anhydride chosen from maleic, fumaric and itaconic acids or anhydrides and
(ii) at least one monomer chosen from vinyl esters, vinyl ethers, vinyl halides, phenylvinyl derivatives, or acrylic acid and its esters, the anhydride functional groups of these copolymers optionally being monoesterified or monoamidated.

Such polymers are disclosed, for instance, in U.S. Pat. Nos. 2,047,398, 2,723,248 and 2,102,113 and GB Patent No. 839 805. Commercial products are, for instance, those sold under the names Gantrez® AN or ES by ISP;

copolymers comprising
(i) at least one anhydride unit chosen from maleic, citraconic and itaconic anhydride units and
(ii) at least one monomer chosen from allyl or methallyl esters, optionally comprising at least one group chosen from acrylamide, methacrylamide, α-olefin, acrylic or methacrylic ester, acrylic or methacrylic acid, and vinylpyrrolidone groups in their chain, the anhydride functional groups of these copolymers optionally being monoesterified or monoamidated.

These polymers are, for example, disclosed in French Patents Nos. 2 350 384 and 2 357 241.

E) Polyacrylamides comprising carboxylate groups.

The homopolymers and copolymers comprising sulpho groups (i.e., derived from sulphonic acid) are, for example, polymers comprising vinylsulphonic, styrenesulphonic, naphthalenesulphonic or acrylamidealkylsulphonic units.

These polymers can be chosen from:

salts of polyvinylsulphonic acid having a molecular mass ranging from 1,000 to 100,000, as well as copolymers with an unsaturated comonomer, such as acrylic or methacrylic acids and their esters, as well as acrylamide or its derivatives, vinyl ethers and vinylpyrrolidone;

salts of polystyrenesulphonic acid, such as the sodium salts sold, for example, under the name Flexan® 500 and Flexan® 130 by National Starch. These compounds are disclosed, for instance, in French Patent FR 2 198 719;

salts of polyacrylamidosulphonic acids, such as those mentioned in U.S. Pat. No. 4,128,631, for example, the polyacrylamidoethylpropanesulphonic acid sold under the name Cosmedia Polymer HSP 1180 by Henkel.

Mention may be made of the anionic branched block polymer sold under the name Fixate G-100 by Noveon, as other anionic fixing polymers which can be used according to the present disclosure.

According to the present disclosure, the anionic fixing polymers may, in at least one embodiment, be chosen from acrylic acid copolymers, such as the acrylic acid/ethyl acrylate/N-tert-butylacrylamide terpolymers sold in particular under the name Ultrahold® Strong by BASF, copolymers derived from crotonic acid, such as the vinyl acetate/vinyl tert-butylbenzoate/crotonic acid terpolymers and the crotonic acid/vinyl acetate/vinyl neododecanoate terpolymers sold, for example, under the name Resin 28-29-30 by National Starch, polymers derived from maleic, fumaric or itaconic acids or anhydrides with vinyl esters, vinyl ethers, vinyl halides, phenylvinyl derivatives, or acrylic acid and its esters, such as the monoesterified methyl vinyl ether/maleic anhydride copolymers sold, for example, under the name Gantrez® by ISP, the copolymers of methacrylic acid and of methyl methacrylate sold under the name Eudragit® L by Rohm Pharma, the copolymers of methacrylic acid and of ethyl acrylate sold under the name Luvimer® MAEX or MAE by BASF, the vinyl acetate/crotonic acid copolymers sold under the name Luviset CA 66 by BASF, the vinyl acetate/crotonic acid copolymers grafted by polyethylene glycol sold under the name Aristoflex® A by BASF or the polymer sold under the name Fixate G-100 by Noveon.

In at least one embodiment, among the anionic fixing polymers mentioned above, one or more of the following polymers may be used: the monoesterified methyl vinyl ether/maleic anhydride copolymers sold under the name Gantrez® ES 425 by ISP, the acrylic acid/ethyl acrylate/N-tert-butylacrylamide terpolymers sold under the name Ultrahold® Strong by BASF, the copolymers of methacrylic acid and of methyl methacrylate sold under the name Eudragit® L by Rohm Pharma, the vinyl acetate/vinyl tert-butyl-benzoate/crotonic acid terpolymers and the crotonic acid/vinyl acetate/vinyl neododecanoate terpolymers sold under the name Resin 28-29-30 by National Starch, the copolymers of methacrylic acid and of ethyl acrylate sold under the name Luvimer® MAEX or MAE by BASF, the vinylpyrrolidone/acrylic acid/lauryl methacrylate terpolymers sold under the name Acrylidone® LM by ISP or the polymer sold under the name Fixate G-100 by Noveon.

Cationic Fixing Polymers

The film-forming cationic fixing polymers which can be used according to the present invention are, in at least one embodiment, chosen from polymers comprising primary, secondary, tertiary and/or quaternary amine groups forming part of the polymer chain or directly connected to the latter and having a molecular weight ranging from 500 to 5,000,000, such as from 1000 to 3,000,000.

In at least one embodiment, the cationic fixing polymers are chosen from homopolymers and copolymers of acrylic and methacrylic esters and amides comprising aminated functional groups, cationic polysaccharides, quaternary copolymers of vinylpyrrolidone and of vinylimidazole, and chitosans.

Mention may be made, among these polymers, of the following cationic polymers:

(1) Homopolymers or copolymers derived from acrylic or methacrylic esters or amides comprising at least one of the units of following formulae: 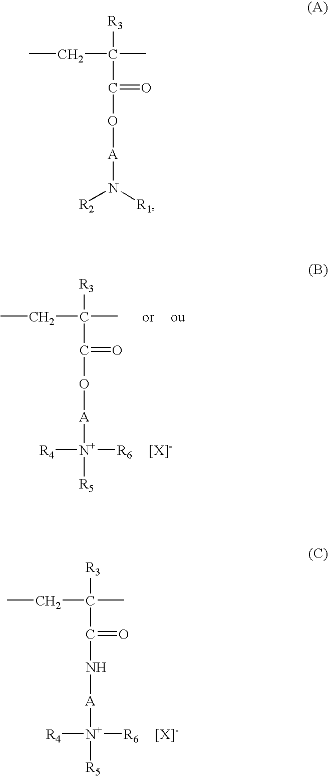
in which

R₃ is chosen from a hydrogen atom and a CH₃ radical;

A is chosen from a linear or branched alkyl group comprising from 1 to 6 carbon atoms and a hydroxyalkyl group comprising from 1 to 4 carbon atoms;

R₄, R₅ and R₆, which are identical or different, are each individually chosen from an alkyl group having from 1 to 18 carbon atoms and a benzyl radical;

R₁ and R₂, which are identical or different, each are individually chosen from a hydrogen atom and an alkyl group having from 1 to 6 carbon atoms;

X is chosen from a methyl sulphate anion and a halide, such as chloride or bromide.

The copolymers of family (1) additionally comprise at least one unit deriving from comonomers which can be chosen from the family of the acrylamides, methacrylamides, diacetone acrylamides, acrylamides and methacrylamides substituted on the nitrogen by lower (C₁-C₄) alkyl groups, groups derived from acrylic or methacrylic acids or their esters, vinyllactams, such as vinylpyrrolidone or vinylcaprolactam, or vinyl esters.

Thus, mention may be made, among these copolymers of family (1), of:

copolymers of acrylamide and of dimethylaminoethyl methacrylate which is quaternized with dimethyl sulphate or with a methyl halide, such as that sold under the name Hercofloc® by Hercules,

copolymers of acrylamide and of methacryloyloxyethyltrimethylammonium chloride, disclosed, for example, in Patent Application EP-A-080 976 and sold under the name Bina Quat P 100 by Ciba-Geigy,

copolymer of acrylamide and of methacryloyloxyethyltrimethylammonium methyl sulphate, such as that sold under the name Reten by Hercules,

vinylpyrrolidone/dialkylaminoalkyl acrylate or methacrylate copolymers, which may or may not be quaternized, such as the products sold under the name “Gafquat®” by ISP, like for example “Gafquat® 734” or “Gafquat® 755”, or the products named “Copolymer® 845, 958 and 937”. These polymers are described in detail in French Patents 2 077 143 and 2 393 573,

polymers comprising a fatty chain and comprising a vinylpyrrolidone unit, such as the products sold under the name Styleze W2O and Styleze W10 by ISP,

dimethylaminoethyl methacrylate/vinylcaprolactam/vinylpyrrolidone terpolymers, such as the product sold under the name Gaffix VC 713 by ISP, and

quaternized vinylpyrrolidone/dimethylaminopropylmethacrylamide copolymers, such as the products sold under the name “Gafquat® HS 100” by ISP.

(2) Cationic polysaccharides, for example comprising quaternary ammonium, such as those disclosed in U.S. Pat. Nos. 3,589,578 and 4,031,307, such as guar gums comprising trialkylammonium cationic groups. Such products are sold, for instance, under the trade names Jaguar C13 S, Jaguar C15 and Jaguar C17 by Meyhall.

(3) Quaternary copolymers of vinylpyrrolidone and of vinylimidazole.

(4) Chitosans or their salts; the salts which can be used are, for example, chitosan acetate, lactate, glutamate, gluconate or pyrrolidonecarboxylate. Mention may made, among these compounds, of the chitosan having a degree of deacetylation of 90.5% by weight sold under the name Kytan Brut Standard by Aber Technologies or the chitosan pyrrolidonecarboxylate sold under the name Kytamer® PC by Amerchol.

(5) Cationic cellulose derivatives, such as the copolymers of cellulose or of cellulose derivatives grafted with a water-soluble monomer comprising a quaternary ammonium and disclosed, for instance, in U.S. Pat. No. 4,131,576, such as hydroxyalkylcelluloses, for example hydroxymethyl-, hydroxyethyl- or hydroxypropylcelluloses, grafted, for example, with a methacryloyloxyethyltrimethylammonium, methacrylamidopropyltrimethylammonium or dimethyidiallylammonium salt.

The marketed products corresponding to this definition include, for example, the products sold under the name “Celquat L 200” and “Celquat H 100” by National Starch.

Amphoteric Fixing Polymers

The amphoteric fixing polymers which can be used in accordance with the present disclosure can be chosen from polymers comprising B and C units distributed randomly in the polymer chain, where B denotes a unit deriving from a monomer comprising at least one basic nitrogen atom and C denotes a unit deriving from an acidic monomer comprising at least one group chosen from carboxyl and sulpho groups or else B and C can denote groups deriving from zwitterionic carboxybetaine or sulphobetaine monomers;

B and C can also denote a cationic polymer chain comprising primary, secondary, tertiary or quaternary amine groups, in which at least one of the amine groups carries a carboxyl or sulpho group connected via a hydrocarbon group, or else B and C form part of a chain of a polymer comprising an α,β-dicarboxyethylene unit, one of the carboxyl groups of which has been reacted with a polyamine comprising at least one primary or secondary amine groups.

In at least one embodiment, the amphoteric fixing polymers are chosen from copolymers comprising acidic vinyl units and comprising basic vinyl units, acylated and crosslinked polyaminoamides, polymers comprising zwitterionic units, polymers derived from chitosan, modified (C₁-C₅)alkyl vinyl ether/maleic anhydride copolymers, amphoteric polyurethanes and amphoteric grafted silicone polymers.

Examples of amphoteric fixing polymers corresponding to the definition given above and used in at least one embodiment herein are chosen from the following polymers:

1) copolymers comprising acidic vinyl units and comprising basic vinyl units, such as those resulting from the copolymerization of a monomer derived from a vinyl compound carrying a carboxyl group, such as acrylic acid, methacrylic acid, maleic acid or α-chloracrylic acid, and of a basic monomer derived from a substituted vinyl compound comprising at least one basic atom, such as dialkylaminoalkyl methacrylate and acrylate or dialkylaminoalkylmethacrylamide and -acrylamide. Such compounds are disclosed, for example, in U.S. Pat. No. 3,836,537.

2) Polymers comprising units deriving:

a) from at least one monomer chosen from acrylamides or methacrylamides substituted on the nitrogen atom by an alkyl group,

b) from at least one acidic comonomer comprising one or more reactive carboxyl groups, and

c) from at least one basic comonomer, such as esters comprising primary, secondary, tertiary and quaternary amine substituents of acrylic and methacrylic acids and the quaternization product of dimethylaminoethyl methacrylate with dimethyl or diethyl sulphate.

In at least one embodiment, the N-substituted acrylamides or methacrylamides used according to the present disclosure are the compounds in which the alkyl groups comprise from 2 to 12 carbon atoms and more particularly N-ethylacrylamide, N-tert-butylacrylamide, N-tert-octylacrylamide, N-octylacrylamide, N-decylacrylamide or N-dodecylacrylamide, and the corresponding methacrylamides.

The acidic comonomers may be, for example, chosen from acrylic, methacrylic, crotonic, itaconic, maleic or fumaric acids and alkyl monoesters having 1 to 4 carbon atoms of maleic or fumaric acids or anhydrides.

The basic comonomers may be, for example, chosen from aminoethyl, butylaminoethyl, N,N′-dimethylaminoethyl and N-tert-butylaminoethyl methacrylates.

Use may be made, in at least one embodiment, of one or more copolymers for which the CTFA name (4th Ed., 1991) is Octylacrylamide/acrylates/butylaminoethyl methacrylate copolymer, such as the products sold under the name Amphomer® or Lovocryl® 47 by National Starch.

(3) Partially or completely acylated and crosslinked polyaminoamides deriving from polyaminoamides of formula: 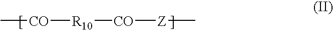
in which

R₁₀ is a divalent group derived from a saturated dicarboxylic acid, from an aliphatic mono- or dicarboxylic acid comprising an ethylenic double bond, from an ester of a lower alkanol having 1 to 6 carbon atoms of these acids, or from a group deriving from the addition of any one of the said acids with a bis-primary or bis-secondary amine, and

Z is a group deriving from a bis-primary, mono- or bis-secondary polyalkylenepolyamine and, in at least one embodiment, represents:

a) in the amount of 60 to 100 mol %, the group 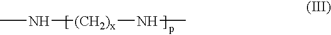
where x=2 and p=2 or 3, or else x=3 and p=2 this group deriving from diethylenetriamine, triethylenetetraamine or dipropylenetriamine;

b) in the amount of 0 to 40 mol %, the above group (III), in which x=2 and p=1 and which derives from ethylenediamine, or the group deriving from piperazine:

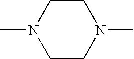

c) in the amount of 0 to 20 mol %, the group —NH—(CH₂)₆—NH— deriving from hexamethylenediamine, these polyaminoamides being crosslinked by addition reaction of a bifunctional crosslinking agent chosen from epihalohydrins, diepoxides, dianhydrides or bis-unsaturated derivatives, by means of 0.025 to 0.35 mol of crosslinking agent per amine group of the polyaminoamide, and acylated by reaction with acrylic acid, chloroacetic acid or an alkanesultone or their salts.

The saturated carboxylic acids may be, for example, chosen from acids having 6 to 10 carbon atoms, such as adipic, 2,2,4-trimethyladipic or 2,4,4-trimethyladipic, or terephthalic acids, and the acids comprising an ethylenic double bond, such as, for example, acrylic, methacrylic or itaconic acids.

The alkanesultones used in the acylation may be propane- or butanesultone and the salts of the acylating agents are, for example, the sodium or potassium salts.

(4) Polymers comprising zwitterionic units of formula: 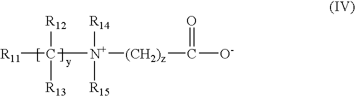
in which

R₁₁ is a polymerizable unsaturated group, such as an acrylate, methacrylate, acrylamide or methacrylamide group,

y and z are each independently an integer from 1 to 3,

R₁₂ and R₁₃ are each independently chosen from a hydrogen atom and from methyl, ethyl and propyl groups, and

R₁₄ and R₁₅ are each independently chosen from a hydrogen atom and an alkyl group such that the sum of the carbon atoms in R₁₄ and R₁₅ does not exceed 10.

The polymers comprising such units can also comprise units derived from non-zwitterionic monomers, such as dimethyl- or diethylaminoethyl acrylate or methacrylate or alkyl acrylates or methacrylates, acrylamides or methacrylamides, or vinyl acetate.

Mention may be made, by way of example, of methyl methacrylate/dimethylcarboxymethylammonioethyl methacrylate copolymers, such as the product sold under the name Diaformer Z301 by Sandoz.

(5) Polymers derived from chitosan comprising monomer units corresponding to the following formulae: 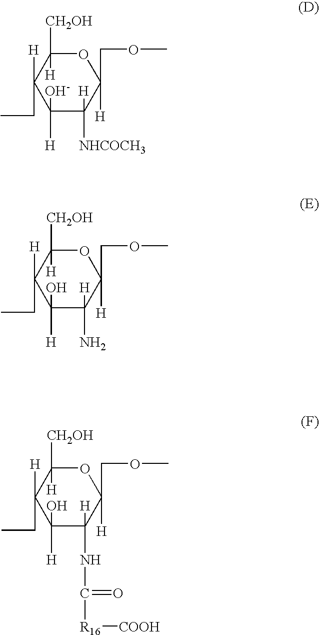
the unit (D) being present in an amount of from 0 to 30%, the unit (E) being present in an amount of from 5 to 50% and the unit (F) being present in an amount of from 30 to 90%, it being understood that, in this unit (F), R₁₆ is a group of formula: 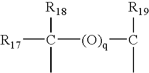
in which, if q=0, R₁₇, R₁₈ and R₁₉, which are identical or different, each are chosen from a hydrogen atom and from residues chosen from methyl, hydroxyl, acetoxy, amino, monoalkylamine, and dialkylamine residues, optionally interrupted by at least one nitrogen atom and/or optionally substituted by at least one group chosen from amine, hydroxyl, carboxyl, alkylthio and sulpho groups, or by an alkylthio residue in which the alkyl group carries an amino residue, at least one of the R₁₇, R₁₈ and R₁₉ groups being, in this case, a hydrogen atom;

or, if q=1, R₁₇, R₁₈ and R₁₉ are each independently chosen from a hydrogen atom, and the salts formed by these compounds with bases or acids.

(6) Polymers corresponding to the formula (V), for example disclosed in French Patent FR 1 400 366: 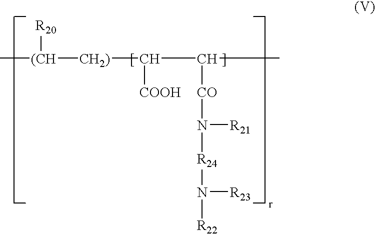
in which

R₂₀ is chosen from a hydrogen atom, a CH₃O, CH₃CH₂O group, and a phenyl group,

R₂₁ is chosen from a hydrogen atom and a lower alkyl group, such as methyl or ethyl,

R₂₂ is chosen from a hydrogen atom and a lower C₁-C₆ alkyl group, such as methyl or ethyl,

R₂₃ is chosen from a lower C₁-C₆ alkyl group, such as methyl or ethyl, and a group corresponding to the formula: —R₂₄—N(R₂₂)₂, wherein R₂₄ is chosen from a —CH₂—CH₂ group, a —CH₂—CH₂—CH₂— group, and a —CH₂—CH(CH₃)— group and R₂₂ having the meanings mentioned above,

R₂₄ is chosen from a —CH₂—CH₂— group, a —CH₂—CH₂—CH₂— group, and a —CH₂—CH(CH₃)— group, and

r is an integer greater or equal to 1.

(7) Polymers derived from the N-carboxyalkylation of chitosan, such as the N-(carboxymethyl)chitosan or the N-(carboxybutyl)chitosan sold under the name “Evalsan” by Jan Dekker.

(8) Amphoteric polymers of the -D-X-D-X— type chosen from:

a) polymers obtained by reaction of chloroacetic acid or sodium chloroacetate with compounds comprising at least one unit of formula:
-D-X-D-X-D- (VI)
where D is a group 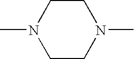
and X denotes the symbol E or E′, E or E′, which are identical or different, denoting a bivalent group which is a straight- or branched-chain alkylene group comprising up to 7 carbon atoms in the main chain which is unsubstituted or substituted by hydroxyl groups and which can additionally comprise oxygen, nitrogen or sulphur atoms or 1 to 3 aromatic and/or heterocyclic rings; the oxygen, nitrogen and sulphur atoms being present in the form of ether, thioether, sulphoxide, sulphone, sulphonium, alkylamine or alkenylamine groups or hydroxyl, benzylamine, amine oxide, quaternary ammonium, amide, imide, alcohol, ester and/or urethane groups.

b) polymers of formula:
-D-X-D-X— (VI′)
where D denotes a group 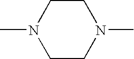
and X denotes the symbol E or E′ and at least once E′, E having the meaning indicated above and E′ being a bivalent group which is a straight- or branched-chain alkylene group having up to 7 carbon atoms in the main chain which is unsubstituted or substituted by at least one hydroxyl group and which comprises at least one nitrogen atom, the at least one nitrogen atom being substituted by an alkyl chain optionally interrupted by an oxygen atom and necessarily comprising at least one carboxyl functional group and at least one hydroxyl functional group and betainized by reaction with chloroacetic acid or sodium chloroacetate.

(9) (C₁-C₅)Alkyl vinyl ether/maleic anhydride copolymers partially modified by semiamidation with an N,N-dialkylaminoalkylamine, such as N,N-dimethylamino-propylamine, or by semiesterification with an N,N-dialkylaminoalkanol. These copolymers can also comprise other vinyl comonomers, such as vinylcaprolactam.

In at least one embodiment of the present disclosure, the amphoteric fixing polymers used one or more chosen from family (3), such as the copolymers with the CTFA name of Octylacrylamide/acrylates/butylaminoethyl methacrylate copolymer, such as the products sold under the names Amphomer®, Amphomer® LV 71 or Lovocryl® 47 by National Starch, and those of family.(4), such as methyl methacrylate/dimethylcarboxy-methylammonioethyl methacrylate copolymers, sold, for example, under the name Diaformer Z301 by Sandoz.

Nonionic Fixing Polymers

The nonionic fixing polymers which can be used according to the present disclosure are chosen, for example, from:

polyalkyloxazolines;

vinyl acetate homopolymers;

vinyl acetate copolymers, such as, for example copolymers of vinyl acetate and of acrylic ester, copolymers of vinyl acetate and of ethylene, or copolymers of vinyl acetate and of maleic ester, for example of dibutyl maleate;

acrylic ester homopolymers and copolymers, such as, for example, copolymers of alkyl acrylates and of alkyl methacrylates, such as the products provided by Rohm & Haas under the names Primal® AC-261 K and Eudragit® NE 30 D, by BASF under the name 8845 or by Hoechst under the name Appretan® N9212;

copolymers of acrylonitrile and of a nonionic monomer chosen, for example, from butadiene and alkyl (meth)acrylates; mention may be made of the products provided under the name CJ 0601 B by Rohm & Haas;

styrene homopolymers;

styrene copolymers, such as, for example, copolymers of styrene and of alkyl (meth)acrylate, such as the products Mowilith® LDM 6911, Mowilith® DM 611 and Mowilith® LDM 6070 provided by Hoechst or the products Rhodopas® SD 215 and Rhodopas® DS 910 provided by Rhone-Poulenc, copolymers of styrene, of alkyl methacrylate and of alkyl acrylate, copolymers of styrene and of butadiene or copolymers of styrene, of butadiene and of vinylpyridine;

polyamides;

vinyllactam homopolymers other than vinylpyrrolidone homopolymers, such as the polyvinylcaprolactam sold under the name Luviskol® Plus by BASF; and

vinyllactam copolymers, such as the poly(vinylpyrrolidone/vinyllactam) copolymer sold under the trade name Luvitec® VPC 55K65W by BASF, poly(vinylpyrrolidone/vinyl acetate) copolymers, such as those sold under the name PVPVA® S630L by ISP or Luviskol® VA 73, VA 64, VA 55, VA 37 and VA 27 by BASF, and poly(vinylpyrrolidone/vinyl acetate/vinyl propionate) terpolymers, such as, for example, that sold under the name Luviskol® VPA 343 by BASF.

The alkyl groups of the nonionic polymers mentioned above may, in at least one embodiment, have from 1 to 6 carbon atoms.

Grafted Silicone Fixing Polymers

According to the present disclosure, it is also possible to use fixing polymers of grafted silicone type comprising a polysiloxane part and a part composed of a silicone-free organic chain, one of the two parts constituting the main chain of the polymer and the other being grafted onto the main chain. The polymers of this type may be amphoteric, anionic or non-ionic. In at least one embodiment, the grafted silicone polymers are anionic or nonionic.

These polymers are disclosed, for example, in Patent Applications EP-A-0 412 704, EP-A-0 412 707, EP-A-0 640 105, WO 95/00578, EP-A-0 582 152 and WO 93/23009 and U.S. Pat. Nos. 4,693,935, 4,728,571 and 4,972,037.

Such polymers are, for example, the copolymers capable of being obtained by radical polymerization from the mixture of monomers formed from:

a) 50 to 90% by weight of tert-butyl acrylate,

b) 0 to 40% by weight of acrylic acid,

c) 5 to 40% by weight of a silicone macromer of formula: 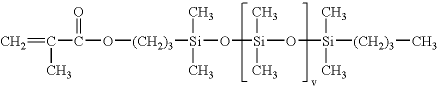
where v is a number ranging from 5 to 700, the percentages by weight being calculated with respect to the total weight of the monomers.

Other examples of grafted silicone polymers are in particular polydimethylsiloxanes (PDMSs) on which are grafted, via a connecting link of thiopropylene type, mixed polymer units of the poly((meth)acrylic acid) type and of the poly(alkyl (meth)acrylate) type and polydimethylsiloxanes (PDMSs) on which are grafted, via a connecting link of thiopropylene type, polymer units of the poly(isobutyl (meth)acrylate) type.

Mention may be made, as another type of silicone fixing polymers, of the product Luviflex® Silk sold by BASF.

Polyurethane Fixing Polymers

Use may also be made, as fixing polymers, of cationic, nonionic, anionic or amphoteric polyurethanes which may or may not be functionalized and which may or may not be silicone-comprising, or their mixtures.

The polyurethanes useful in at least some embodiments of the present disclosure are those disclosed in Applications EP 0 751 162, EP 0 637 600, EP 0 648 485 and FR 2 743 297, assigned to L'Oreal, and in Applications EP 0 656 021 and WO 94/03510 of BASF and EP 0 619 111 of National Starch.

Mention may be made, as polyurethanes suitable in at least one embodiment of the present disclosure, of the products sold under the names Luviset PUR® and Luviset® Si PUR by BASF.

When the composition according to the present disclosure comprises at least one additional fixing polymer, the concentration of the at least one polymer ranges from 0.1 to 20%, for example from 0.5 to 10%, by weight with respect to the total weight of the composition.

The compositions according to the present disclosure can also comprise, as additional cosmetic adjuvant, at least one additional thickening polymer, also referred to as “rheology-adjusting agent”, other than the modified guar gums.

The rheology-adjusting agents can be chosen from fatty acid amides (coconut diethanol- or monoethanolamide, oxyethylenated alkyl ether carboxylic acid monoethanolamide), cellulose thickeners (hydroxyethylcellulose, hydroxypropylcellulose, carboxymethylcellulose), gums of microbial origin (xanthan gum, scleroglucan gum), crosslinked homopolymers of acrylic acid or of acrylamidopropanesulphonic acid, and associative polymers.

The associative polymers which can be used herein are water-soluble polymers capable, in an aqueous medium, of reversibly associating with one another or with other molecules. Their chemical structure comprises hydrophilic regions and hydrophobic regions characterized by at least one fatty chain.

The associative polymers which can be used herein can be of anionic, cationic, amphoteric or nonionic type.

The concentration of additional thickening polymer(s) ranges from 0.01 to 20%, such as from 0.05 to 10%, by weight with respect to the total weight of the composition.

The compositions according to the present disclosure can also comprise, as additional cosmetic adjuvant, at least one compound chosen from silicones, silicone-comprising fatty substances and silicone-free fatty substances.

The silicones which can be used in the compositions according to the present disclosure can be linear, cyclic, branched or unbranched and volatile or nonvolatile. They can be in the soluble, dispersed or microdispersed form and can be provided in the form of oils, resins or gums. They can, for example, be polyorganosiloxanes which are insoluble in the cosmetically acceptable medium.

Organopolysiloxanes are defined in more detail in the work by Walter Noll, “Chemistry and Technology of Silicones” (1968), Academic Press. They can be volatile or nonvolatile.

When they are volatile, the silicones are chosen, for example, from those having a boiling point ranging from 60° C. to 260° C. and, in at least one embodiment, from:

(i) cyclic silicones comprising from 3 to 7 silicon atoms, such as from 4 to 5 silicon atoms. They are, for example, octamethylcyclotetrasiloxane, sold, for instance, under the name of “Volatile Silicone 7207” by Union Carbide or “Silbione 70045 V 2” by Rhodia, decamethylcyclopentasiloxane, sold under the name of “Volatile Silicone 7158” by Union Carbide or “Silbione 70045 V 5” by Rhodia, and their mixtures.

Mention may also be made of cyclocopolymers of the dimethylsiloxane/methylalkylsiloxane type, such as “Silicone Volatile FZ 3109”, sold by Union Carbide, with the chemical structure:

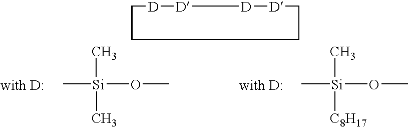

Mention may also be made of mixtures of cyclic silicones with silicon-derived organic compounds, such as the mixture of octamethylcyclotetrasiloxane and of tetratrimethylsilylpentaerythritol (50/50) and the mixture of octamethylcyclotetrasiloxane and of 1,1′-oxy(2,2,2′,2′,3,3′-hexatrimethylsilyloxy)bisneopentane;

(ii) linear volatile silicones having 2 to 9 silicon atoms and having a viscosity of less than or equal to 5×10⁻⁶ m²/s at 25° C. Such silicones include, for example, decamethyltetrasiloxane, sold in particular under the name “SH 200” by Toray Silicone. Silicones coming within this class are also described in the article published in Cosmetics and Toiletries, Vol. 91, Jan. 76, p. 27-32, Todd & Byers, “Volatile Silicone Fluids for Cosmetics”.

Mention may, for example, be made, among nonvolatile silicones, of polyalkylsiloxanes, polyarylsiloxanes, polyalkylarylsiloxanes, silicone gums and resins, polyorganosiloxanes modified by organofunctional groups, and their mixtures.

The organomodified silicones which can be used in accordance with the present disclosure are silicones as defined above comprising, in their structure, at least one organofunctional group attached via a hydrocarbon group.

Mention may be made, among organomodified silicones, of polyorganosiloxanes comprising:

polyethyleneoxy and/or polypropyleneoxy groups optionally comprising C₆-C₂₄ alkyl groups, such as the products named dimethicone copolyol sold by Dow Corning under the name DC 1248 or the oils Silwet® L 722, L 7500, L 77 or L 711 from Union Carbide and the (C₁₂)alkyl methicone copolyol sold by Dow Corning under the name Q2 5200;

substituted or unsubstituted aminated groups, such as the products sold under the names GP 4 Silicone Fluid and GP 7100 by Genesee or the products sold under the names Q2 8220 and Dow Corning 929 or 939 by Dow Corning. The substituted aminated groups are in particular C₁-C₄ aminoalkyl groups;

thiol groups, such as the products sold under the names “GP 72 A” and “GP 71” from Genesee;

alkoxylated groups, such as the product sold under the name “Silicone Copolymer F-755” by SWS Silicones and Abil Wax® 2428, 2434 and 2440 by Goldschmidt;

hydroxylated groups, such as the polyorganosiloxanes comprising a hydroxyalkyl functional group disclosed in French Patent Application FR-A-85 16334;

acyloxyalkyl groups, such as, for example, the polyorganosiloxanes disclosed in U.S. Pat. No. 4,957,732;

anionic groups of the carboxylic acid type, such as, for example, in the products disclosed in Patent EP 186 507 from Chisso Corporation, or of the alkylcarboxyl type, such as those present in the product X-22-3701 E from Shin-Etsu; 2-hydroxyalkylsulphonate type; or 2-hydroxyalkyl thiosulphate type, such as the products sold by Goldschmidt under the names “Abil® S201” and “Abil® S255”;

hydroxyacylamino groups, such as the polyorganosiloxanes disclosed in Application EP 342 834. Mention may be made, for example, of the product Q2-8413 from Dow Corning.

The silicone oils which can be used in the compositions according to the present disclosure are volatile or nonvolatile polymethylsiloxanes comprising a linear or cyclic silicone chain which are liquid or pasty at ambient temperature, for example cyclopolydimethylsiloxanes (cyclomethicones), such as cyclohexasiloxane; polydimethylsiloxanes comprising pendant alkyl, alkoxy or phenyl groups or alkyl, alkoxy or phenyl groups at the end of the silicone chain, which groups have from 2 to 24 carbon atoms; phenylated silicones, such as phenyl trimethicones, phenyl dimethicones, phenyl-(trimethylsiloxy)diphenylsiloxanes, diphenyl dimethicones, diphenyl(methyidiphenyl)trisiloxanes, (2-phenylethyl)trimethylsiloxysilicates or polymethyl-phenylsiloxanes; and their mixtures.

The silicone gums which can be used in the compositions according to the present disclosure are polydiorganosiloxanes of high molecular mass, i.e., ranging from 200,000 to 2,000,000,.used alone or as a mixture in a solvent chosen from volatile silicones, polydimethylsiloxane oils, polymethylphenylsiloxane oils, polydiphenyl-dimethylsiloxane oils, isoparaffins, methylene chloride, pentane, hydrocarbons and their mixtures.

In at least one embodiment, use is made of a silicone gum with a molecular weight of less than 1,500,000. The silicone gums are, for example, polydimethylsiloxanes, polyphenylmethylsiloxanes, poly(diphenylsiloxane/dimethylsiloxane)s, poly(dimethyl-siloxane/methylvinylsiloxane)s, poly(dimethylsiloxane/phenylmethylsiloxane)s or poly(diphenylsiloxane/dimethylsiloxane/methylvinylsiloxane)s.

These silicone gums can be terminated at the chain end by trimethylsilyl or dimethylhydroxysilyl groups.

The silicone resins which can be used in the compositions according to the present disclosure are crosslinked siloxane systems including the units R₂SiO_{2/2}, RSiO_{3/2} and SiO_{4/2} in which R is chosen from a hydrocarbon group having from 1 to 6 carbon atoms and from a phenyl group. In at least one embodiment, R is chosen from a lower (C₁-C₆) alkyl radical and a phenyl radical.

The silicone-free fatty substances which can be used in the compositions according to the present disclosure are all the organic or inorganic, natural or synthetic, silicone-free oils, waxes or resins.

An oil within the meaning of the present disclosure is a lipophilic compound which is liquid at ambient temperature (approximately 25° C.) and which exhibits a reversible solid/liquid change in state. Animal and vegetable oils comprise, as essential constituents, triesters of propane-1,2,3-triol.

Mention may be made, as oils which can be used in the composition of the present disclosure, for example of:

hydrocarbon oils of animal origin, such as perhydrosqualene;

hydrocarbon oils of vegetable origin, such as liquid triglycerides of fatty acids comprising from 4 to 10 carbon atoms, such as triglycerides of heptanoic or octanoic acids, or also, for example, sunflower,.maize, soybean, cucumber, grape seed, sesame, hazelnut, apricot, macadamia, arara, castor or avocado oils, triglycerides of caprylic/capric acids, such as, for example, those sold by Stearineries Dubois or those sold under the names Miglyol 810, 812 and 818 by Dynamit Nobel, jojoba oil or shea butter oil;

synthetic esters and ethers, such as of fatty acids, such as the oils of formulae R⁶COOR⁷ and R⁶OR⁷ in which R⁶ is the residue of a fatty acid comprising from 8 to 29 carbon atoms and R⁷ is a branched or unbranched hydrocarbon chain comprising from 3 to 30 carbon atoms, such as, for example, purcellin oil, isononyl isononanoate, isopropyl myristate, 2-ethylhexyl palmitate, 2-octyldodecyl stearate, 2-octyidodecyl erucate or isostearyl isostearate; hydroxylated esters, such as isostearyl lactate, octyl hydroxystearate, octyldodecyl hydroxystearate, diisostearyl malate or triisocetyl citrate; heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate;

linear or branched hydrocarbons of mineral or synthetic origin, such as volatile or nonvolatile liquid paraffins and their derivatives, liquid petrolatum, polydecenes or hydrogenated polyisobutene, such as parleam oil;

fluid fatty alcohols having from 8 to 26 carbon atoms, such as octyldodecanol, 2-butyloctanol, oleyl alcohol, linoleyl alcohol or linolenyl alcohol;

alkoxylated, such as ethoxylated, fatty alcohols, such as oleth-12;

partially hydrocarbon-comprising fluorinated oils, such as those disclosed in the document JP-A-2 295912. Mention may also be made, as fluorinated oils, of perfluoromethylcyclopentane and perfluoro(1,3-dimethylcyclohexane), for example sold under the names “Flutec PC1®” and “Flutec PC3®” by BNFL Fluorochemicals; perfluoro(1,2-dimethylcycobutane); perfluoroalkanes, such as dodecafluoropentane and tetradecafluorohexane, for example sold under the names “PF 5050®” and “PF 5060®” by 3M, or else bromoperfluorooctane, for example sold under the name “Foralkyl®” by Atochem; nonafluoromethoxybutane, for example sold under the name “MSX 4518®” by 3M, and nonafluoroethoxyisobutane; or perfluoromorpholine derivatives, such as 4-(trifluoromethyl)perfluoromorpholine, for example sold under the name “PF 5052®” by 3M.

The term “hydrocarbon oil” in the list of the oils mentioned above is understood to mean any oil comprising predominantly carbon and hydrogen atoms and optionally ester, ether, fluoro, carboxylic acid and/or alcohol groups.

A wax within the meaning of the present disclosure is a lipophilic compound which is solid at ambient temperature (approximately 25° C.), which exhibits a reversible solid/liquid change in state, which has a melting point of greater than approximately 40° C. which can range up to 200° C. and which exhibits an anisotropic crystalline arrangement in the solid state. Animal and vegetable waxes comprise, as essential constituents, esters of carboxylic acids and of long-chain alcohols. Generally, the size of the crystals of the wax is such that the crystals diffract and/or scatter light, conferring a more or less opaque cloudy appearance on the composition which comprises them. On bringing the wax to its melting point, it is possible to render it miscible with oils and to form a microscopically homogeneous mixture but, on bringing the temperature of the mixture back to ambient temperature, recrystallization of the wax in the oils of the mixture is obtained, which recrystallization can be detected microscopically and macroscopically (opalescence).

Mention may be made, as waxes which can be used in the present disclosure, of waxes of animal origin, such as beeswax, spermaceti, lanolin wax and lanolin derivatives; vegetable waxes, such as sunflower, rice or apple peel waxes, carnauba wax, candelilla wax, ouricury wax, Japan wax, cocoa butter or cork fibre or sugarcane waxes; mineral waxes, for example paraffin wax, petrolatum wax, lignite wax or microcrystalline waxes, ceresin or ozokerite; synthetic waxes, such as polyethylene waxes or Fischer-Tropsch waxes; and their mixtures.

The concentration of compound(s) chosen from silicones, silicone-comprising fatty substances and silicone-free fatty substances can range from 0.01 to 20%, such as from 0.05 to 10%, by weight with respect to the total weight of the composition.

The styling composition according to the present disclosure can additionally comprise at least one additive chosen from nonionic, anionic, cationic and amphoteric surface-active agents, additional nonionic, anionic, cationic and amphoteric polymers other than the fixing polymers used in the compositions according to the invention, ceramides and pseudoceramides, vitamins and provitamins, including panthenol, water-soluble and fat-soluble sunscreen-agents which may or may not comprise silicone, solid fillers and particles, such as, for example, inorganic and organic pigments which may or may not be coloured, pearlescent and opacifying agents, glitter, active particles, dyes, sequestering agents, plasticizing agents, solubilizing agents, acidifying agents, basifying agents, neutralizing agents, inorganic and organic thickening agents, antioxidants, hydroxy acids, penetrating agents, fragrances and preservatives.

A person skilled in the art will take care to choose the optional additives and their amounts so that they do not harm the properties of the compositions disclosed herein.

These additives can be present in the composition according to the present disclosure in an amount ranging from 0 to 20% by weight, with respect to the total weight of the composition.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

The following examples illustrate the present invention and should not be regarded in any way as limiting the invention.

### EXAMPLES

The following formulations A and B were prepared

| | as % of |
|---|---|
| | active material |
| Formulation A | |
| Diglycol/CHDM/Isophthalates/SIP copolymer | 20 |
| (Eastman AQ 55 S - Eastman) | |
| Hydroxypropyl guar (Jaguar HP 105 - Rhodia) | 1.5 |
| Carbomer (Carbopol Ultrez 10 - Noveon) | 0.25 |
| Deionized water | q.s. 100 |
| Fragrance and ingredients | q.s. for |
| Formulation B | |
| Diglycol/CHDM/Isophthalates/SIP copolymer | 10 |
| (Eastman AQ 55 S - Eastman) | |
| Hydroxypropyl guar (Jaguar HP 105 - Rhodia) | 2 |
| Deionized water | q.s. 100 |
| Fragrance and ingredients | q.s. for |

Eastman AQ 55 S, sold by Eastman, is a diethylene glycol/1,4-cyclohexanedimethanol/isophthalate/sulphoisophthalate copolymer.

Procedure:

2 g of the formulation to be tested were applied to a lock of natural hair weighing 2.7 g and with a length of 27 cm.

The treated lock was wound around a roller with a diameter of 1 cm in order to give it a shape.

The combination was allowed to dry in the air and then the lock was gently unwound from the roller.

The locks thus shaped were subsequently immersed in a salt water bath (3% NaCl) with a volume of 8 liters at ambient temperature with magnetic stirring at 100 revolutions/minute.

The length of the lock over time was measured in order to evaluate the form retention of the hair shaping.

Measurements of the Form Retention of the Hair Shaping:

Form retention of the hair shaping in %: (Lᵢ−L)/(Lᵢ−L₀)*100

L: Length of the curled lock at time t

L₀: Length of the curled lock after hair shaping and removing from the roller

Lᵢ: Length of the lock before hair shaping on the roller

Form retention of the hair shaping in %

| Immersion time | | A | B |
|---|---|---|---|
| 0 | | 100 | 100 |
| 25 | seconds | 100 | 100 |
| 30 | seconds | 100 | 100 |
| 35 | seconds | 100 | 100 |
| 40 | seconds | 100 | 100 |
| 10 | minutes | 100 | 100 |

What is claimed is:

1. A cosmetic composition comprising, in a cosmetically acceptable medium, at least one water-dispersible linear sulphonic polyester and at least one modified guar gum.

2. A cosmetic composition according to claim 1, wherein the at least one water-dispersible linear sulphonic polyester is a polycondensate of at least one dicarboxylic acid or an ester thereof, of at least one diol, and of at least one difunctional sulphoaryldicarboxyl compound substituted on the aromatic ring by an —SO₃M group wherein M is chosen from a hydrogen atom and a metal ion.

3. A cosmetic composition according to claim 2, wherein M is a metal ion chosen from Na⁺, Li⁺ and K⁺.

4. A cosmetic composition according to claim 1, wherein the at least one water-dispersible linear sulphonic polyester is obtained from isophthalic acid, sodium salt of sulphoisophthalic acid, diethylene glycol and 1,4-cyclohexanedimethanol.

5. A cosmetic composition according to claim 1, wherein the Tg of the at least one water-dispersible linear sulphonic polyester is greater than or equal to 50° C.

6. A cosmetic composition according to claim 1, wherein the at least one water-dispersible linear sulphonic polyester is present in an amount ranging from 0.1 to 40% by weight with respect to the total weight of the composition.

7. A cosmetic composition according to claim 6, wherein the at least one water-dispersible linear sulphonic polyester is present in an amount ranging from 1 to 30% by weight with respect to the total weight of the composition.

8. A cosmetic composition according to claim 7, wherein the at least one water-dispersible linear sulphonic polyester is present in an amount ranging from 5 to 25% by weight with respect to the total weight of the composition.

9. A cosmetic composition according to claim 1, wherein the at least one modified guar gum is chosen from at least one guar gum alkylated by at least one C₁₋₈ alkyl group, at least one guar gum hydroxyalkylated by at least one C₁₋₈ hydroxyalkyl group and at least one guar gum acylated by at least one C₁₋₈ alkyl group.

10. A cosmetic composition according to claim 9, wherein the at least one modified guar gum is a hydroxypropylated guar gum.

11. A cosmetic composition according to claim 1, wherein the at least one modified guar gum is present in an amount ranging from 0.05 to 10% by weight with respect to the total weight of the composition.

12. A cosmetic composition according to claim 11, wherein the at least one modified guar gum is present in an amount ranging from 0.1 to 5% by weight with respect to the total weight of the composition.

13. A cosmetic composition according to claim 12, wherein the at least one modified guar gum is present in an amount ranging from 0.5 to 3% by weight with respect to the total weight of the composition.

14. A cosmetic composition according to claim 1, wherein the at least one water-dispersible linear sulphonic polyester and the at least one modified guar gum are present in a ratio of from 0.01 to 100.

15. A cosmetic composition according to claim 14, wherein the ratio of the at least one water-dispersible linear sulphonic polyester to the at least one modified guar gum ranges from 1 to 75.

16. A cosmetic composition according to claim 14, wherein the ratio of the at least one water-dispersible linear sulphonic polyester to the at least one modified guar gum ranges from 2 to 50.

17. A cosmetic composition according to claim 1, further comprising at least one additional fixing polymer chosen from anionic, cationic, amphoteric and nonionic fixing polymers and mixtures thereof.

18. A composition according to claim 17, wherein the cationic fixing polymers are chosen from homopolymers and copolymers of acrylic and methacrylic esters and amides comprising aminated functional groups, cationic polysaccharides, quaternary copolymers of vinylpyrrolidone and of vinylimidazole, and chitosans.

19. A composition according to claim 17, wherein the anionic fixing polymers are chosen from homopolymers and copolymers of acrylic and methacrylic acid and their salts, copolymers of crotonic acid, copolymers of C₄-C₈ monounsaturated carboxylic acids and anhydrides, polyacrylamides comprising carboxylate groups, homopolymers and copolymers comprising sulpho groups, anionic polyurethanes and anionic grafted silicone polymers.

20. A composition according to claim 17, wherein the amphoteric fixing polymers-are chosen from-copolymers comprising acidic vinyl units and comprising basic vinyl units, acylated and crosslinked polyaminoamides, polymers comprising zwitterionic units, polymers derived from chitosan, modified (C₁-C₅)alkyl vinyl ether/maleic anhydride copolymers amphoteric polyurethanes and amphoteric grafted silicone polymers.

21. A composition according to claim 17, wherein the nonionic fixing polymers are chosen from polyalkyloxazolines, vinyl acetate homopolymers and copolymers, acrylic ester homopolymers and copolymers, acrylonitrile copolymers, styrene homopolymers and copolymers, polyamides, vinyllactam homopolymers other than vinylpyrrolidone homopolymers, vinyllactam copolymers, nonionic polyurethanes and nonionic grafted silicone polymers.

22. A cosmetic composition according to claim 17, wherein the at least one additional fixing polymer is present in an amount ranging from 0.1 to 20% by weight with respect to the total weight of the composition.

23. A cosmetic composition according to claim 22, wherein the at least one additional fixing polymer is present in an amount ranging from 0.5 to 10% by weight with respect to the total weight of the composition.

24. A cosmetic composition according to claim 1, further comprising at least one additional thickening polymer other than the at least one modified guar gum.

25. A cosmetic composition according to claim 24, wherein the at least one additional thickening polymer is present in an amount ranging from 0.01 to 20%.

26. A cosmetic composition according to claim 25, wherein the at least one additional thickening polymer is present in an amount ranging from 0.05 to 10% by weight with respect to the total weight of the composition.

27. A cosmetic composition according to claim 1, further comprising at least one compound chosen from silicones, silicone-comprising fatty substances and silicone-free fatty substances.

28. A cosmetic composition according to claim 27, wherein the at least one compound chosen from silicones, silicone-comprising fatty substances and silicone-free fatty substances is present in an amount ranging from 0.01 to 20% by weight relative to the total weight of the composition.

29. A cosmetic composition according to claim 28, wherein the at least one compound chosen from silicones, silicone-comprising fatty substances and silicone-free fatty substances is present in an amount ranging from 0.05 to 10% by weight relative to the total weight of the composition.

30. A cosmetic composition according to claim 1, further comprising at least one additive chosen from nonionic, anionic, cationic and amphoteric surface-active agents, additional nonionic, anionic, cationic and amphoteric polymers, ceramides and pseudoceramides, vitamins and provitamins, water-soluble and fat-soluble sunscreen agents which may or may not comprise silicone, solid fillers and particles, pearlescent and opacifying agents, glitter, active particles, dyes, sequestering agents, plasticizing agents, solubilizing agents, acidifying agents, basifying agents, neutralizing agents, inorganic and organic thickening agents, antioxidants, hydroxy acids, penetrating agents, fragrances and preservatives.

31. A cosmetic composition according to claim 30, wherein said solid fillers and particles are chosen from inorganic and organic pigments which may or may not be colored.

32. A method for shaping, fixing, and/or form retention of a hairstyle comprising applying to hair a cosmetic composition comprising, in a cosmetically acceptable medium, at least one water-dispersible linear sulphonic polyester and at least one modified guar gum.

33. A method according to claim 32, wherein the at least one water-dispersible linear sulphonic polyester is obtained from isophthalic acid, sodium salt of sulphoisophthalic acid, diethylene glycol and 1,4-cyclohexanedimethanol.

34. A method according to claim 32, wherein the at least one modified guar gum is a hydroxypropylated guar gum.

35. A method for creating a water-resistant hairstyle, comprising applying to hair a cosmetic composition comprising, in a cosmetically acceptable medium, at least one water-dispersible linear sulphonic polyester and at least one modified guar gum.
